(19) United States
(12) Patent Application Publication
(10) Pub. No.: US 2017/0311863 A1
(43) Pub. Date: Nov. 2, 2017

(54) EMOTION ESTIMATION DEVICE AND EMOTION ESTIMATION METHOD

(71) Applicant: OMRON Corporation, Kyoto-shi (JP)

(72) Inventor: Jumpei MATSUNAGA, Kusatsu-shi (JP)

(73) Assignee: OMRON Corporation, Kyoto-shi (JP)

(21) Appl. No.: 15/652,866

(22) Filed: Jul. 18, 2017

Related U.S. Application Data
(63) Continuation of application No. PCT/JP2015/086237, filed on Dec. 25, 2015.

(30) Foreign Application Priority Data
Feb. 13, 2015 (JP) 2015-026336

(51) Int. Cl. A61B 5/16; A61B 5/00; G06K 9/00

(52) U.S. Cl. CPC A61B 5/165; G06K 9/00302; A61B 5/0077; G06K 9/00268; A61B 2576/00

(57) ABSTRACT

An emotion estimation device includes: an image obtaining unit that obtains plural images in which an object person is photographed in time series; an expression recognizer that recognizes an expression of the object person from each of the plural images obtained by the image obtaining unit; a storage in which expression recognition results of the plural images are stored as time-series data; and an emotion estimator that detects a feature associated with a time change of the expression of the object person from the time-series data stored in the storage in an estimation target period, and estimates the emotion of the object person in the estimation target period based on the detected feature.

### CROSS-REFERENCE TO RELATED APPLICATION

This application is a continuation application of International Application No. PCT/JP2015/086237, filed on Dec. 25, 2015, which claims priority based on the Article 8 of Patent Cooperation Treaty from prior Japanese Patent Application No. 2015-026336, filed with the Japan Patent Office on Feb. 13, 2015, the entire contents of which are incorporated herein by reference.

### FIELD

The disclosure relates to a technology of estimating a person's emotion from a facial expression.

### BACKGROUND

Not only a method for communicating verbally with others but also communication (also referred to as nonverbal communication) using means other than words are used in communication with others. Examples of the nonverbal communication include a facial expression, a look, a gesture, and a tone of voice, and play an important role for understanding the emotion of the other party. Nowadays, an attempt to use the nonverbal communication in a man-machine interaction is performed. Among others, emotion estimation based on the facial expression is expected to be an elemental technology necessary for implementation of the advance communication between the person and the machine.

Conventionally, many methods are proposed as a technology of recognizing the facial expression from an image, and some of the methods already come into practical use. For example, JP 2007-65969 A discloses an algorithm that extracts shape features (Fourier descriptors) of eyes and a mouth from the image and calculates an index indicating degrees of six expressions (happiness, surprise, fear, anger, disgust, and sadness) based on the shape features.

However, even if the facial expression can be recognized from the image, the person's emotion (mental state) is not easily estimated from a recognition result of the facial expression. Because usually the expression changes in various ways during the communication, the person's emotion cannot correctly be understood only from the facial expression in one image. As what is called a poker face or an artificial smile shows, a real intention (real emotion) is not always appears on a face.

### SUMMARY

An aspect of the present invention has been made in consideration of the circumstances mentioned above, an object thereof is to provide a technology of being able to accurately estimate the person's emotion based on the facial expression recognized from the image.

A configuration, in which a feature associated with a time change of the facial expression of the object person is detected from time-series data of the facial expression to estimate the emotion of the object person based on the detected feature, is adopted in one or more embodiments of the present invention in order to achieve the object.

Specifically, an emotion estimation device configured to estimate an emotion of an object person, the emotion estimation device includes: an image obtaining unit configured to obtain plural images in which the object person is photographed in time series; an expression recognizer configured to recognize an expression of the object person from each of the plural images obtained by the image obtaining unit; a storage in which expression recognition results of the plural images are stored as time-series data; and an emotion estimator configured to detect a feature associated with a time change of the expression of the object person from the time-series data stored in the storage in an estimation target period, and estimate the emotion of the object person in the estimation target period based on the detected feature.

Accordingly, because one aspect of the present invention pays attention to the feature associated with the time change of the facial expression in the estimation target period, the change, reaction, and display of the emotion can be captured in the estimation target period, and an estimation result can be obtained with higher accuracy and reliability compared with the case that the estimation is performed only by the facial expression in one image.

It may be preferable that, when detecting a change of a kind of a main expression persistently expressed on a face of the object person as the feature associated with the time change of the expression, the emotion estimator estimates the emotion corresponding to the changed kind of the main expression to be the emotion of the object person in the estimation target period. Frequently a person consciously or unconsciously shows the expression when the emotion (mental state) changes. Accordingly, the change of the kind of the main expression has a strong causal relationship with the change of the person's emotion, and at least the changed main expression has a high probability of reflecting the emotion of the object person. Therefore, the emotion of the object person can more correctly be understood by paying attention to the change of the kind of the main expression.

It may be preferable that, when detecting appearance of a microexpression expressed for an instant on a face of the object person as the feature associated with the time change of the expression, the emotion estimator estimates the emotion corresponding to a kind of the expression expressed as the microexpression to be the emotion of the object person in the estimation target period. The microexpression means an expression that appears and vanishes instantly on the face like a flash. For example, when a person tries to hide intentionally the expression or create an untrue expression such that the other party does not notice the real emotion of the person, frequently the real emotion appears as the microexpression. Therefore, the emotion of the object person can more correctly be understood by paying attention to the appearance of the microexpression.

It may be preferable that, when detecting both a change of a kind of a main expression persistently expressed on a face of the object person and appearance of a microexpression expressed for an instant on a face of the object person as the feature associated with the time change of the expression, the emotion estimator estimates the emotion in which the emotion corresponding to the changed kind of the main expression and the emotion corresponding to a kind of the expression expressed as the microexpression are compounded to be the emotion of the object person in the estimation target period. Thus, the complicated emotion or real emotion of the object person can be expected to be understood by paying attention to both the change of the kind of the main expression and the appearance of the microexpression.

It may be preferable that, when detecting both a change of a kind of a main expression persistently expressed on a face of the object person as the feature associated with the time change of the expression, and when detecting appearance of a microexpression expressed for an instant on a face of the object person in a transition period in which the kind of the main expression changes as the feature associated with the time change of the expression, the emotion estimator estimates the emotion in which the emotion corresponding to the changed kind of the main expression and the emotion corresponding to a kind of the expression expressed as the microexpression are compounded to be the emotion of the object person in the estimation target period. For example, in the case that the object person intentionally hides the real emotion, frequently the facial expression change in which the real emotion is hidden behind another expression is observed after the real emotion appears instantly as the microexpression. That is, it is probably said that the microexpression appearing in the transition period of the main expression expresses the real emotion of the object person. Therefore, the real emotion of the object person can be expected to be understood by paying attention to the microexpression appearing in the transition period of the main expression.

It may be preferable that the expression recognizer calculates a score in which each degree of a plurality of kinds of the expressions is digitized from the image of the object person, and outputs the score of each expression as the expression recognition result, and when a maximum state of the score of one expression continues for a predetermined time or more in the plurality of kinds of the expressions, the emotion estimator determines that the one expression is the main expression. Accordingly, the facial expression and main expression of the object person can be estimated in quantitative and objective manners. A minute expression change such as a noise is ignored, so that the reliability of the estimation can be improved.

It may be preferable the expression recognizer calculates a score in which each degree of a plurality of kinds of the expressions is digitized from the image of the object person, and outputs the score of each expression as the expression recognition result, and when the score of a certain expression exceeds a threshold for an instant, the emotion estimator determines that the expression is the microexpression. According to the configuration, the facial expression and microexpression of the object person can be estimated in quantitative and objective manners. For example, when the score of a certain expression exceeds a threshold from a state lower than the threshold and returns to the state lower than the threshold again for an instant, the emotion estimator may determine that the expression is the microexpression. For example, the instant may mean a time of 1 second or less.

One or more embodiments of the present invention can also be understood as an emotion estimation device including at least a part of the configurations or functions. One or more embodiments of the present invention can also be understood as an emotion estimation method including at least a part of the pieces of processing, a program causing a computer to perform the emotion estimation method, or a computer-readable recording medium in which the program is non-transiently stored. One or more embodiments of the present invention can be implemented by a combination of the configurations or the pieces of processing as long as technical inconsistency is not generated.

In one or more embodiments of the present invention, the person's emotion can accurately be estimated based on the facial expression recognized from the image.

### BRIEF DESCRIPTION OF THE DRAWINGS

FIG. 1 is a view illustrating a configuration example of an emotion estimation device;

FIG. 2 is a flowchart illustrating a flow of emotion estimation processing;

FIG. 3 is a view illustrating an example of time-series data of an expression recognition result stored in a storage;

FIGS. 4A to 4C are views illustrating examples of the time-series data and main expression change detection; and

FIG. 5 is a view illustrating an example of the time-series data and microexpression detection.

### DETAILED DESCRIPTION

Hereinafter, embodiments of the present invention will be described in detail with reference to the drawings. However, unless otherwise noted, the present invention is not limited to sizes, materials, shapes, and relative dispositions of components described in the following embodiment.

(Device Configuration)

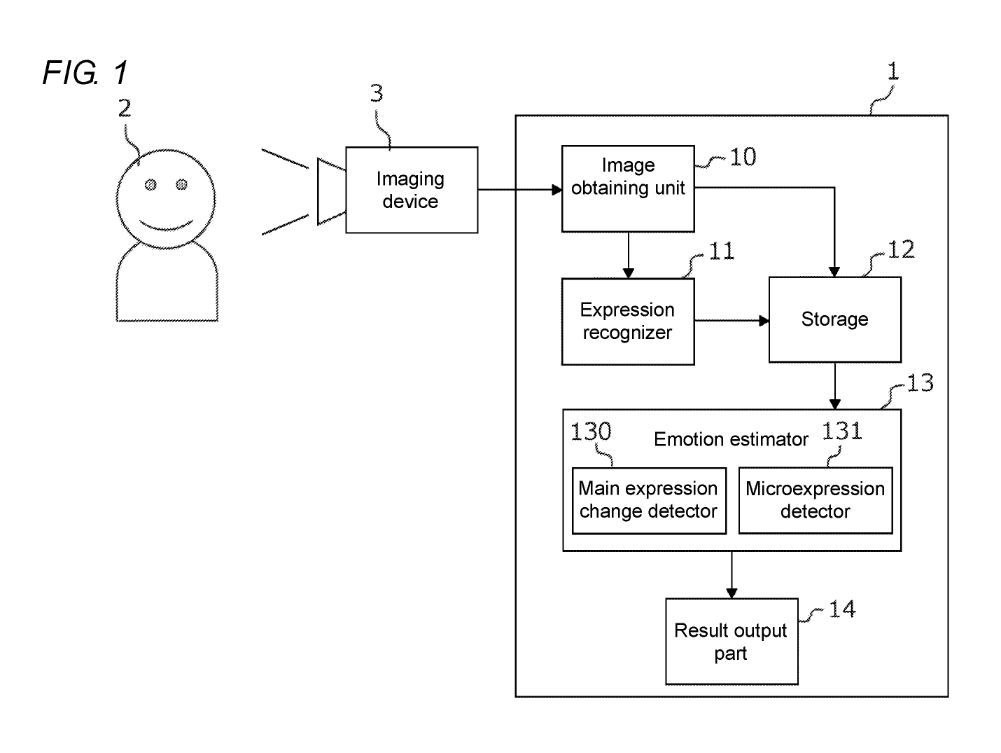

FIG. 1 is a view illustrating a configuration example of an emotion estimation device according to an embodiment of the present invention. An emotion estimation device 1 analyzes an image in which an object person 2 is photographed, and estimates emotions of the object person 2. The emotion estimation device 1 can be used as a module that implements the man-machine interaction using the nonverbal communication. For example, such advanced control that the robot adaptively change operation while seeing user's reaction can be performed, when the emotion estimation device 1 is mounted on a home robot that performs domestic affairs and assistance. Additionally, the emotion estimation device can be applied to every industrial field such as artificial intelligence, computers, smartphones, tablet terminals, game machines, home electric appliances, industrial machines, and automobiles.

The emotion estimation device 1 in FIG. 1 includes an image obtaining unit 10, an expression recognizer 11, a storage 12, an emotion estimator 13, and a result output part 14 as a main configuration. The emotion estimator 13 further includes a main expression change detector 130 and a microexpression detector 131.

The image obtaining unit 10 has a function of obtaining an image from an imaging device 3. In performing the emotion estimation, plural images (for example, 20 fps continuous images), in which a face of the object person 2, are sequentially captured from the imaging device 3. The imaging device 3 is constructed with a monochrome or color camera. In FIG. 1, the imaging device 3 is provided separately from the emotion estimation device 1. Alternatively, the imaging device 3 may be mounted on the emotion estimation device 1. The expression recognizer 11 has a function of recognizing a facial expression from the image through image sensing processing. The storage 12 has a function of storing an expression recognition result output from the expression recognizer 11 as time-series data. The emotion estimator 13 has a function of detecting a feature associated with a time change of an expression of the object person 2 from the time-series data stored in the storage 12, and estimating an emotion of the object person 2 based on the detected feature. The result output part 14 has a function of outputting an emotion estimation result of the emotion estimator 13 (such as display of the emotion estimation result on a display device and transmission of information on the emotion estimation result to an external device).

The emotion estimation device 1 can be constructed with a computer including a CPU (processor), a memory, an auxiliary storage device, an input device, an display device, and a communication device. A program stored in the auxiliary storage device is loaded on the memory, and the CPU executes the program, thereby implementing each function of the emotion estimation device 1. However, a part of or all the functions of the emotion estimation device 1 can also be implemented by a circuit such as an ASIC and an FPGA. Alternatively, a part of the functions (for example, the functions of the expression recognizer 11, storage 12, and emotion estimator 13) of the emotion estimation device 1 may be implemented by cloud computing or distributed computing.

(Emotion Estimation Processing)

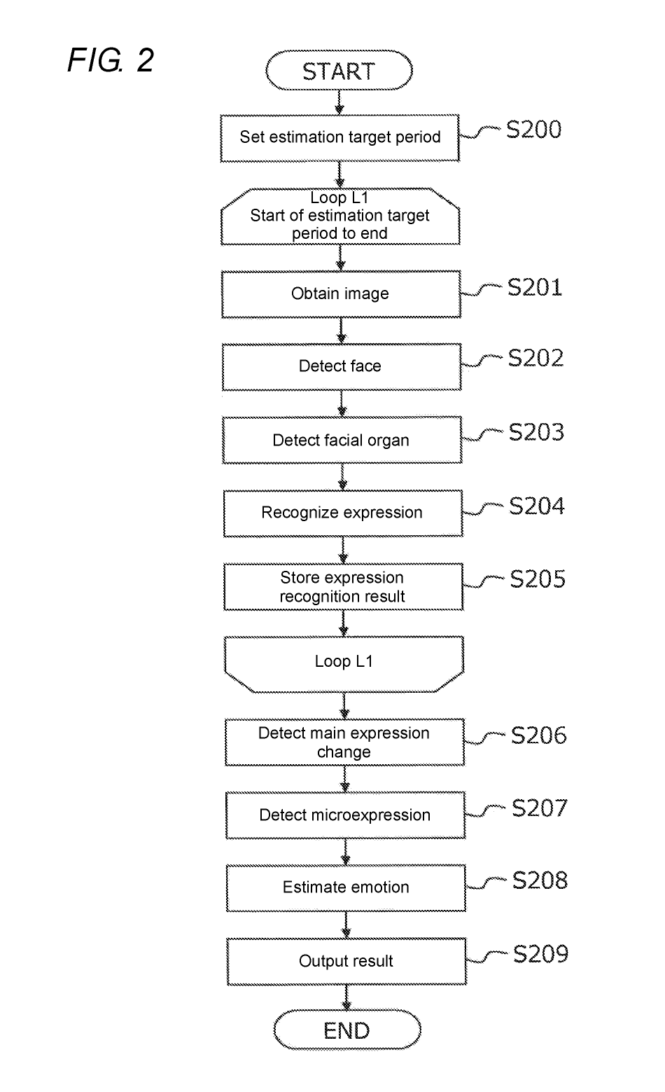

A flow of emotion estimation processing performed by the emotion estimation device 1 will be described with reference to FIG. 2. FIG. 2 is a flowchart illustrating the flow of the emotion estimation processing.

A period (referred to as a estimation target period) becoming an emotion estimation target is set in Step S200. The emotion estimation device 1 may automatically set the estimation target period, the external device or external software that uses the emotion estimation result may assign estimation target period to the emotion estimation device 1, or a user may manually set the estimation target period. The estimation target period can arbitrarily be set, preferably the estimation target period is set to time lengths of several seconds to several tens of seconds. Possibly an emotion change cannot be detected when the estimation target period is set excessively short, and the emotion estimation result is hardly narrowed because of too many emotion changes when the estimation target period is set excessively long. For example, a period of several seconds to several tens of seconds including an event generation clock time may be set to the estimation target period to know a person's reaction to a certain event (such as machine operation, conversation output, and service provision).

The subsequent pieces of processing in Steps S201 to S205 are repeatedly performed, for example, every 50 milliseconds (corresponds to 20 fps) from a start to an end of the estimation target period (loop L1).

In Step S201, the image obtaining unit 10 obtains the image in which the object person 2 is photographed from the imaging device 3. Desirably the image in which a front face of the object person 2 is photographed as much as possible is obtained in order to estimate the emotion based on the facial expression. Then, the expression recognizer 11 detects the face from the image (Step S202), and detects a facial organ (such as eyes, eyebrows, a nose, and a mouth) (Step S203). Because any algorithm including a well-known technique may be used in the face detection and the facial organ detection, the detailed description is omitted.

The expression recognizer 11 recognizes the facial expression of the object person 2 using detection results in Steps S202 and S203 (Step S204). A kind of the facial expression is expressed by a word indicating the emotion. The recognition of the expression means that the kind of the facial expression is identified, namely, that the kind of the facial expression that is of a recognition target is specified by the word indicating the emotion. At this point, the facial expression may be specified by the word indicating a single emotion or a combination of words indicating emotions. For the combination of the words indicating the emotions, the word indicating each emotion may be weighted. In the embodiment, based on an expression analysis of Paul Ekman, the facial expression is classified into seven kinds including “straight face”, “happiness”, “anger”, “disgust”, “surprise”, “fear”, and “sadness”. A score is output as the expression recognition result such that degrees (expression-likeness, also referred to as an expression degree) of the seven kinds of the expressions become 100 in total. The score of each expression is also referred to as an expression component value.

Any algorithm including a well-known technique may be used in the expression recognition in Step S204. An example of the expression recognition processing will be described below. The expression recognizer 11 extracts a feature amount associated with the relative position or shape of the facial organ based on position information on the facial organ. For example, a Haar-like feature amount, a distance between feature points, and the Fourier descriptor disclosed in JP 2007-65969 A can be used as the feature amount. The feature amount extracted by the expression recognizer 11 is input to a classifier for each of the seven kinds of the facial expressions to calculate the degrees of the expressions. Each classifier can be generated by learning in which a sample image is used. Finally, the expression recognizer 11 performs normalization such that output values of the seven classifiers become 100 in total, and outputs the score (expression component value) of the seven kinds of the expressions.

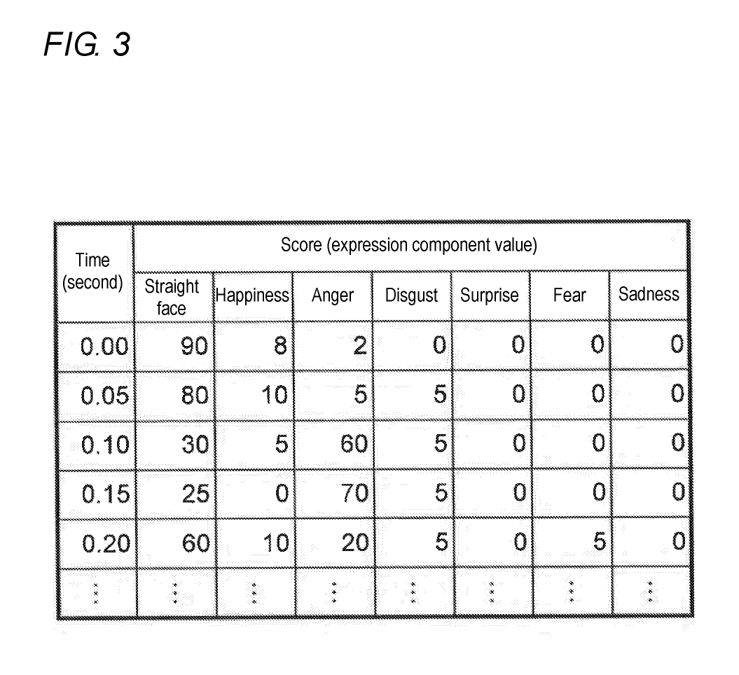

The expression recognizer 11 stores the expression recognition result in a database of the storage 12 together with time stamp information (Step S205). FIG. 3 illustrates an example of the time-series data of the expression recognition result stored in the storage 12, and illustrates the expression recognition result of each 50-millisecond line.

When the time-series data of the expression recognition result is obtained in the estimation target period through the above processing, the emotion estimator 13 performs the emotion estimation processing. As illustrated in FIG. 2, the emotion estimation processing of the embodiment is constructed with three steps, namely, main expression change detection (Step S206), microexpression detection (Step S207), and emotion estimation (Step S208). Each step will be described in detail below.

(1) Main Expression Change Detection (Step S206)

The main expression change detection is processing of detecting the change of the kind of the expression (referred to as a main expression) that comes out persistently on the face of the object person 2 as the feature associated with the time change of the facial expression. The term “persistently” means that, when generally a person observes the expression, the person feels that the expression continues for a persistent time. For example, the persistent time that the person feels is 3 seconds or more. The term “comes out” means that generally a person can make an observation to recognize the expression. Any expression classification algorithm can be adopted so long as the algorithm outputs a result approximate to an observation result of a person. When the person changes the emotion (mental state), frequently the person consciously or unconsciously shows the expression. Accordingly, the change of the kind of the main expression has a strong causal relationship with the emotion change, and it is considered that at least the changed main expression has a high probability of reflecting the emotion of the object person 2. Therefore, the emotion of the object person 2 can more correctly be understood by paying attention to the change of the kind of the main expression.

In the embodiment, in order to quantitatively and objectively evaluate the main expression, the “main expression” is defined as “the expression has the largest score in the seven kinds of the expressions and its state continues for a predetermined time or more”. The “predetermined time” can arbitrarily be set, and desirably the “predetermined time” is set to several seconds to several tens of seconds in consideration of a general time for which the identical expression is persistent (in the embodiment, the “predetermined time” is set to 3 seconds). The main expression is not limited to the above definition. For example, reliability of the main expression determination can be enhanced by adding a condition that “the score of the main expression is larger than a predetermined value” or a condition that “a score difference between the main expression and another expression is larger than or equal to a predetermined value”.

The main expression change detector 130 reads the time-series data from the storage 12 to check whether the main expression having the score matched with the above definition exists. The main expression change detector 130 outputs information indicating whether the main expression is detected and information indicating whether the kind of the main expression changes in the estimation target period (when the main expression can be detected) as the detection result.

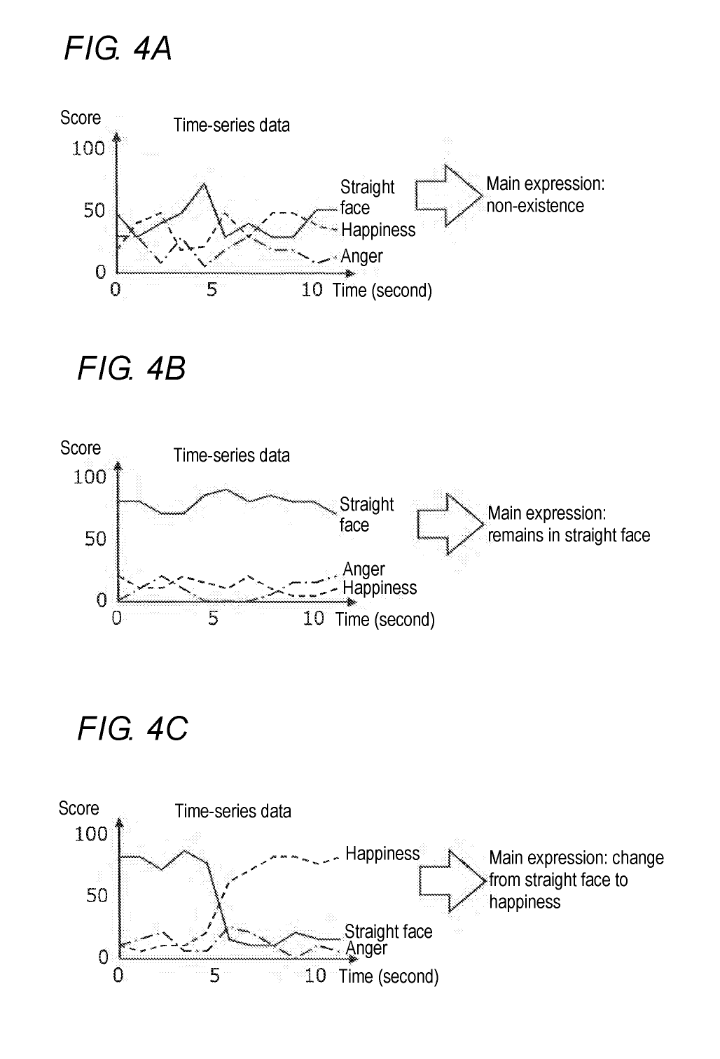

FIGS. 4A to 4C are views illustrating the time-series data and the detection result. In FIGS. 4A to 4C, a horizontal axis indicates the time, a vertical axis indicates the score, and each graph indicates the time change of the score of the expression (because the expressions except for the straight face, happiness, and anger have the score equal to nearly zero, the expressions are not illustrated). In the example of FIG. 4A, there is no expressing having the extremely large score, and the main expression does not exist because a magnitude correlation among the scores of the expressions changes frequently. Accordingly, the main expression change detector 130 outputs the detection result of “main expression: non-existence”. In the example of FIG. 4B, the “straight face” maintains the maximum score throughout the estimation target period. Accordingly, the main expression change detector 130 outputs the detection result of “main expression: remains in “straight face””. In the example of FIG. 4C, the “straight face” has the maximum score for about 5 seconds in a first half of the estimation target period, and the “happiness” has the maximum score for about 5 seconds in a second half of the estimation target period. Accordingly, the main expression change detector 130 outputs the detection result of “main expression: change from “straight face” to “happiness””.

In the case that the expression is not fixed as illustrated in FIG. 4A, or in the case that the expression does not change as illustrated in FIG. 4B, the emotion of the object person 2 is hardly estimated from the expression. On the other hand, in the case that the expression change is clearly recognized in the middle of the estimation target period as illustrated in FIG. 4C, there is a high probability that reaction (emotion) of the object person 2 to some sort of event generated immediately before or in the first half of the estimation target period is expressed as the main expression in the second half of the estimation target period. Therefore, in the embodiment, the detection result of “the change of the kind of the main expression” is used in the emotion estimation.

(2) Microexpression Detection (Step S207)

The microexpression detection means processing of detecting the appearance of the expression (referred to as a microexpression) that comes out on the face of the object person 2 for an instant as the feature associated with the time change of the facial expression. The term “for an instant” means generally a person observes the expression within a time for which the person feels an instant. For example, the time for which the person feels an instant is 1 second or less. The term “comes out” has the meaning identical to that of the main expression. For example, when a person tries to hide intentionally the expression or create an untrue expression such that the other party does not notice the real emotion of the person, frequently the real emotion appears as the microexpression. Therefore, the emotion of the object person 2 can more correctly be understood by paying attention to the appearance of the microexpression.

In the embodiment, in order to quantitatively and objectively evaluate the microexpression, the “microexpression” is defined as “the score exceeds a threshold for an instant”. For example, a criterion of the instant may be set to 1 second or less. The “threshold” can arbitrarily be set. For example, the threshold may be set to about 30 to about 70. It is reported that generally many microexpressions vanish within 200 milliseconds. Therefore, in the embodiment, the criterion of the instant is set to 200 milliseconds. The threshold of the score is set to 50. Accordingly, the microexpression detector 131 of the embodiment determines the expression to be the “microexpression” in the case that “the score of a certain expression exceeds 50 from the state lower than 50 and returns to the state lower than 50 within 200 milliseconds”.

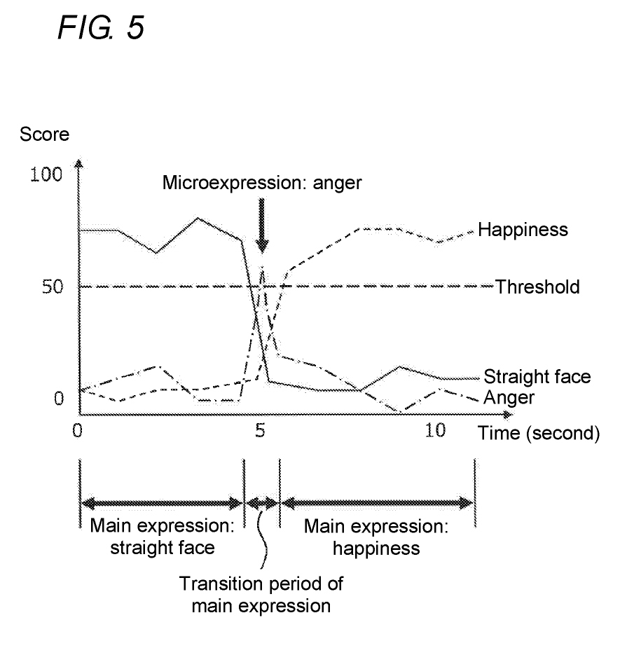

The microexpression detector 131 reads the time-series data from the storage 12 to check whether the microexpression having the score matched with the above definition exists. In the embodiment, because the expression recognition result is obtained every 50 milliseconds, the determination of the microexpression may be made when the score exceeding 50 continues at least one time or three times or less. FIG. 5 illustrates an example in which the microexpression of “anger” is detected at a time point of about 5 seconds in the estimation target period.

In the case that a person tries to hide intentionally the real expression, the facial expression change that the real emotion is hidden behind another expression after appearing for an instant as the microexpression is frequently observed. For example, in the case that the microexpression “anger” appears in a transition period in which the main expression changes from the “straight face” to the “happiness” as illustrated in FIG. 5, it is considered that, although the object person 2 has a slightly negative emotion at heart, the object person 2 smiles (be delighted) such that the slightly negative emotion does not appears on the face as the expression. Thus, the microexpression appearing in the transition period of the main expression is the information necessary for the understanding of the real emotion of the object person 2. Accordingly, for the microexpression detection, a whole of the estimation target period is not set to a detection range, but only the transition period of the main expression may be set to the detection range. When the detection range is restricted to the transition period of the main expression, the time necessary for the processing of detecting the microexpression can be shortened, and the microexpression strongly associated with the emotion of the object person 2 can be extracted.

(3) Emotion Estimation (Step S208)

The emotion estimator 13 estimates the emotion of the object person 2 (Step S208) based on the detection results of the main expression change detection (Step S206) and microexpression detection (Step S207).

Specifically, the emotion estimation of the object person 2 is performed by the following rule.
In the case that the change of the kind of the main expression is detected while the microexpression is not detected: the emotion estimator 13 estimates the emotion corresponding to the kind of the changed main expression to be the emotion of the object person 2 in the estimation target period. In the example of FIG. 4C, the object person 2 has the emotion of “happiness”. At this point, the score of the expression is added as information indicating the degree (magnitude) of the emotion, and the emotion may be expressed like “80% of happiness”.
In the case that the microexpression is detected while the change of the kind of the main expression is not detected: the emotion estimator 13 estimates the emotion corresponding to the kind of the detected microexpression to be the emotion of the object person 2 in the estimation target period. Similarly the score of the expression may be added as the information indicating the degree of the emotion.
In the case that both the change of the kind of the main expression and the microexpression are detected: the emotion estimator 13 estimates the emotion in which the emotion corresponding to the kind of the changed main expression and the emotion corresponding to the microexpression are compounded to be the emotion of the object person 2 in the estimation target period. In the example of FIG. 5, the changed main expression is the “happiness”, and the microexpression is the “anger”. Therefore, for example, the emotion of the object person 2 is estimated to be “happiness but slight discontent”. Alternatively, a microexpression point of the “anger” may be subtracted from the score of the “happiness” to output the estimation result of “60% of happiness”.
In the case that neither the change of the kind of the main expression nor the microexpression are detected: the emotion estimator 13 return an error because the emotion estimation cannot be performed based on the facial expression.

When the emotion estimation result is obtained, the result output part 14 outputs the emotion estimation result (Step S209). When a robot or a computer is controlled based on the emotion estimation result, the advanced communication between the person and the machine can be expected to be implemented such that “identical action continues because the other party looks happy”, or such that “another ideal is proposed because the other party feels discontent”.

The configuration of the embodiment has the following advantages.

Because the emotion estimation device 1 pays attention to the feature associated with the time change of the facial expression in the estimation target period, the change, reaction, and display of the emotion can be captured in the estimation target period, and the estimation result can be obtained with higher accuracy and reliability compared with the case that the estimation is performed only by the facial expression in one image. Particularly the emotion of the object person can more correctly be understood by paying attention to the features that are of the change of the kind of the main expression and the appearance of the microexpression. Additionally, in the case that both the change of the kind of the main expression and the microexpression are detected, the estimation is performed while the change of the kind of the main expression and the microexpression are compounded, so that the complicated emotion or real emotion of the object person can be expected to be understood.

The configuration of the embodiment illustrates only one specific example of the present invention, but does not limited to the scope of the present invention. Various specific configurations can be made without departing from the scope of the present invention. For example, both the main expression change detection (Step S206) and the microexpression detection (Step S207) are performed in the embodiment. Alternatively, only one of the main expression change detection and the microexpression detection may be performed. In the embodiment, the seven-kind expression classification is used. Alternatively, another expression classification may be used.

1. An emotion estimation device configured to estimate an emotion of an object person, the emotion estimation device comprising:
an image obtaining unit configured to obtain a plurality of images in which the object person is photographed in time series;
an expression recognizer configured to recognize an expression of the object person from each of the plurality of images obtained by the image obtaining unit;
a storage in which expression recognition results of the plurality of images are stored as time-series data; and
an emotion estimator configured to detect a feature associated with a time change of the expression of the object person from the time-series data stored in the storage in an estimation target period, and estimate the emotion of the object person in the estimation target period based on the detected feature.

2. The emotion estimation device according to claim 1, wherein, when detecting a change of a kind of a main expression persistently expressed on a face of the object person as the feature associated with the time change of the expression, the emotion estimator estimates the emotion corresponding to the changed kind of the main expression to be the emotion of the object person in the estimation target period.

3. The emotion estimation device according to claim 1, wherein, when detecting appearance of a microexpression expressed for an instant on a face of the object person as the feature associated with the time change of the expression, the emotion estimator estimates the emotion corresponding to a kind of the expression expressed as the microexpression to be the emotion of the object person in the estimation target period.

4. The emotion estimation device according to claim 1, wherein, when detecting both a change of a kind of a main expression persistently expressed on a face of the object person and appearance of a microexpression expressed for an instant on a face of the object person as the feature associated with the time change of the expression, the emotion estimator estimates the emotion in which the emotion corresponding to the changed kind of the main expression and the emotion corresponding to a kind of the expression expressed as the microexpression are compounded to be the emotion of the object person in the estimation target period.

5. The emotion estimation device according to claim 1, wherein, when detecting both a change of a kind of a main expression persistently expressed on a face of the object person as the feature associated with the time change of the expression, and when detecting appearance of a microexpression expressed for an instant on a face of the object person in a transition period in which the kind of the main expression changes as the feature associated with the time change of the expression, the emotion estimator estimates the emotion in which the emotion corresponding to the changed kind of the main expression and the emotion corresponding to a kind of the expression expressed as the microexpression in the transition period are compounded to be the emotion of the object person in the estimation target period.

6. The emotion estimation device according to claim 2, wherein the expression recognizer calculates a score in which each degree of a plurality of kinds of the expressions is digitized from the image of the object person, and outputs the score of each expression as the expression recognition result, and
when a maximum state of the score of one expression continues for a predetermined time or more in the plurality of kinds of the expressions, the emotion estimator determines that the one expression is the main expression.

7. The emotion estimation device according to claim 3, wherein the expression recognizer calculates a score in which each degree of a plurality of kinds of the expressions is digitized from the image of the object person, and outputs the score of each expression as the expression recognition result, and
when the score of a certain expression exceeds a threshold for an instant, the emotion estimator determines that the expression is the microexpression.

8. The emotion estimation device according to claim 7, wherein, when the score of a certain expression exceeds a threshold from a state lower than the threshold and returns to the state lower than the threshold again for an instant, the emotion estimator determines that the expression is the microexpression.

9. The emotion estimation device according to claim 3, wherein the instant means a time of 1 second or less.

10. An emotion estimation method for estimating an emotion of an object person using a computer, the emotion estimation method comprising the steps of:
obtaining a plurality of images in which the object person is photographed in time series;
recognizing an expression of the object person from each of the plurality of images obtained by the image obtaining unit;
storing expression recognition results of the plurality of images in a storage as time-series data; and
detecting a feature associated with a time change of the expression of the object person from the time-series data stored in the storage in an estimation target period, and estimating the emotion of the object person in the estimation target period based on the detected feature.

11. A non-transitory computer-readable recording medium storing a program causing a computer to perform operations comprising the steps of the emotion estimation method according to claim 10.
